(19) United States
(12) Patent Application Publication
(10) Pub. No.: US 2005/0107441 A1
(43) Pub. Date: May 19, 2005

(54) Compositions and methods for altering glucose production

(76) Inventors: Steven Anthony Kliewer, Dallas, TX (US); Bryan James Goodwin, Durham, NC (US); James Michael Way, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/499,545

(22) PCT Filed: Dec. 18, 2002
(86) PCT No.: PCT/US02/40360

Related U.S. Application Data
(60) Provisional application No. 60/344,876, filed on Dec. 21, 2001.

(51) Int. Cl. A61K 49/00; A61K 31/4439; A61K 31/426

(52) U.S. Cl. 514/340; 424/9.1; 514/369

(57) ABSTRACT

Methods for identifying agents which alter glucose production by modulating expression and/or activity of the nuclear receptor short heterodimer partner or SHP are provided. Also provided are compositions containing such agents and methods of using such agents to alter glucose production in a subject.

### FIELD OF THE INVENTION

The present invention relates to methods and compositions for altering glucose production via agents that modulate expression and/or activity of the nuclear receptor short heterodimer partner or SHP. Compositions of the present invention comprising agents that induce or increase expression and/or activity of SHP decrease glucose production. Accordingly, these compositions are expected to be useful in treatment of hyperglycemia. Compositions of the present invention comprising agents that inhibit or decrease expression and/or activity of SHP increase glucose production. Accordingly, these compositions are expected to useful in treatment of hypoglycemia.

### BACKGROUND OF THE INVENTION

Insulin has a number of important physiological effects in the liver including the suppression of glucose production, also referred to as gluconeogenesis (O'Brien, R. M., and Granner, D. K. Physiol. Rev. 1996 76:1109-61).

Insulin is known to repress the transcription of several genes involved in gluconeogenesis including phosphoenolpyruvate carboxykinase (PEPCK), which catalyzes the rate limiting step in gluconeogenesis (O'Brien, R. M., and Granner, D. K. Physiol. Rev. 1996 76:1109-61;

Pilkis, S. J., and Granner, D. K. Annu. Rev. Physiol. 1992 54:885-909). Thus, compounds that repress PEPCK expression and consequently decrease hepatic glucose production may be useful in the treatment of diabetes (both Type 1 and Type 2) and other conditions characterized by elevated glucose production and hyperglycemia.

The PEPCK gene is regulated by hepatocyte nuclear factor 4 (HNF4; Hall et al. Proc. Natl. Acad. Sci. USA 1995 92:412-6; Yoon et al. Nature 2001 413:131-8). HNF4 is an established binding partner of short heterodimer partner (SHP; Lee et al. Mol. Cell Biol. 2000 20:187-95).

Short heterodimer partner (SHP) is an orphan member of the nuclear receptor family of transcription factors (Seol et al. Science 1996 272:1336-9). SHP is unusual in that it lacks the DNA binding domain typically found in nuclear receptors. SHP regulates the transcription of target genes by interacting with other nuclear receptors including HNF4 and liver receptor homolog 1 (LRH-1) and suppressing their activity (Goodwin et al. Mol. Cell. 2000 6:517-26; Lee et al. Mol. Cell Biol. 2000 20:187-95; Lu et al. Mol. Cell 2000 6:507-15). Recently, it was demonstrated that SHP expression is induced in the liver by the bile acid receptor FXR (Goodwin et al. Mol. Cell. 2000 6:517-26; Lee et al. Mol. Cell Biol. 2000 20:187-95; Lu et al. Mol. Cell 2000 6:507-15). FXR binds as a heterodimer with the 9-cis retinoic acid receptor (RXR) to a DNA response element in the SHP promoter. The induction of SHP expression by FXR results in the suppression of several genes including CYP7A1. CYP7A1 catalyzes the rate-limiting step in the classical pathway for the conversion of cholesterol to bile acids (Goodwin et al. Mol. Cell. 2000 6:517-26; Lu et al. Mol. Cell 2000 6:507-15). SHP represses CYP7A1 by interacting with LRH-1, which binds to and stimulates the transcription of CYP7A1 (Goodwin et al. Mol. Cell. 2000 6:517-26; Lu et al. Mol. Cell 2000 6:507-15). This nuclear receptor cascade provides a feedback regulatory mechanism for bile acids to coordinately suppress the transcription of genes involved in bile acid synthesis.

CYP7A1 expression is also repressed by insulin (Subbiah, M. T., and Yunker, R. L. Biochem. Biophys. Res. Commun. 1984 124:896-902), although the molecular mechanism remained unclear.

It has now been found that SHP expression is also induced by the hormone insulin in the livers of Zucker diabetic fatty rats, a standard rodent model of diabetes. Conversely, PEPCK expression has been shown to be repressed under these same conditions. Thus, these data are indicative of SHP induction being responsible for the inhibition of PEPCK expression and gluconeogenesis in the liver. Thus, agents that induce SHP expression and/or activity are expected to be useful in the treatment of diseases associated with overproduction of glucose such as hyperglycemia while agents that inhibit SHP expression and/or activity are expected to be useful in the treatment of diseases associated with underproduction of glucose such as hypoglycemia.

### SUMMARY OF THE INVENTION

An object of the present invention is to provide methods for identifying new therapeutic agents that alter gluconeogenesis or the production of glucose via modulation of the expression and/or activity of SHP. In one embodiment, agents of the present invention induce expression and/or activity of SHP, thereby inhibiting production of glucose. Such agents are useful in the treatment of conditions relating to overproduction of glucose such as hyperglycemia. In another embodiment, agents of the present invention inhibit expression and/or activity of SHP, thereby inducing production of glucose. Such agents are useful in the treatment of conditions relating to underproduction of glucose such as hypoglycemia.

Another object of the present invention is to provide compositions for use in altering glucose production, said compositions comprising an agent that modulates SHP expression and/or activity.

Another object of the present invention is to provide a method for altering glucose production in a subject in need thereof, which comprises administering to the subject an agent that modulates the expression and/or activity of SHP. By administering an agent that induces or increases the expression and/or activity of SHP, glucose production in the subject is inhibited. By administering an agent that inhibits or decreases the expression and/or activity of SHP, glucose production in the subject in induced.

Another object of the present invention is to provide a method for treating hyperglycemia in a subject comprising administering to a subject suffering from hyperglycemia an agent that induces or increases expression and/or activity of SHP.

Yet another object of the present invention is to provide a method for treating hypoglycemia in a subject comprising administering to a subject suffering from hypoglycemia an agent that inhibits or decreases expression and/or activity of SHP.

### DETAILED DESCRIPTION OF THE INVENTION

Hyperglycemia, or abnormally high levels of glucose in the circulating blood, occurs in individuals suffering from various conditions including, but not limited to, obesity, impaired glucose tolerance, insulin resistance, metabolic syndrome X, Type 2 diabetes, Type 1 diabetes, and cardiovascular disease. Sulfonylureas are the most widely prescribed oral drugs for treating hyperglycemia. Sulfonylureas act by stimulating the pancreas to secrete more insulin. Metformin, a member of the biguanide family is also used clinically and lowers hyperglycemia by other mechanisms. Competitive inhibitors of intestinal brush-border alpha-glucosidase and potentiators of insulin such as the thiazolidiones are also sometimes used as oral antihyperglycemic drugs.

Hypoglycemia, or an abnormally small concentration of glucose in the circulating blood, can occur in certain endocrine disorders such as hypopituitarism, Addison's disease, and myxedema, in disorders relating to liver malfunction, in instances of renal failure, and in pancreatic cancer. Increasing blood glucose levels can alleviate the symptoms associated with these hypoglycemic disorders.

The present invention relates to new compositions and methods for altering glucose production via alteration in expression and/or activity of the nuclear receptor short heterodimer partner or SHP.

Using a standard rodent model for diabetes, the Zucker diabetic fatty (ZDF) rats, it was demonstrated that SHP expression is induced by the hormone insulin. In contrast, under the same conditions phosphoenolpyruvate carboxykinase (PEPCK), which catalyzes the rate limiting step in gluconeogenesis (O'Brien, R. M., and Granner, D. K. Physiol. Rev. 1996 76:1109-61; Pilkis, S. J., and Granner, D. K. Annu. Rev. Physiol. 1992 54:885-909) was repressed.

In these experiments, ZDF rats were treated for 6 hours with insulin or vehicle (0.9% normal saline) alone. At the end of this period, the rats were killed and RNA prepared from their livers. Real-time quantitative PCR analysis of pooled RNA samples revealed that SHP mRNA levels increased approximately 17 fold in response to insulin. Conversely, PEPCK levels decreased by approximately 2 fold in response to insulin.

These data are consistent with SHP repressing transcription of the PEPCK gene. Further, these data are indicative of SHP induction being responsible for the inhibition of PEPCK expression and gluconeogenesis in the liver.

Thus, the present invention provides new methods for identifying compositions for treatment of hyperglycemia and hypoglycemia based upon the ability of a test agent to modulate SHP expression and/or activity. Test agents that induce or increase SHP expression and/or activity are expected to be useful in the treatment of diseases associated with overproduction of glucose while test agents that inhibit or decrease SHP expression and/or activity are expected to be useful in the treatment of diseases associated with the underproduction of glucose. Accordingly, the present invention also relates to compositions comprising agents that modulate SHP expression and/or activity as well as methods of using these agents to alter glucose production in a subject.

For purposes of the present invention, by “modulation”, “modulate”, or “modulator” it is meant to regulate, adjust or alter physiological conditions or parameters associated with SHP. Thus, examples of modulation include, but are not limited to, an agent either increasing or decreasing gene expression or activity of the SHP, alterations in timing of expression of this nuclear receptor, increases or decrease in glucose production or gluconeogenesis, and alterations in glucose homeostasis. In a preferred embodiment of the present invention, SHP expression and/or activity is induced or increased, thereby resulting in decreased expression and/or activity of PEPCK and decreased glucose production.

By “alter”, “altering” or “alteration” for purposes of the present invention, it is meant that glucose levels are increased or decreased upon administration of a modulator of SHP expression and/or activity as compared to glucose levels prior to administration of the modulator.

By “agent” or “test agent” for purposes of the present invention, it is meant to be inclusive of any molecule that increases or decreases SHP suppression of PEPCK. In a preferred embodiment, the agent is a small organic molecule other than insulin. It is also preferred that the molecule be a ligand for SHP.

Various assays for identifying ligands of SHP can be used.

For example, ligands for use in the compositions of the present invention can be identified routinely through screening of libraries of compounds using assays such as the FRET assay as described in Parks, D. J. 1999. Science 284:1365-1368 and in WO 00/25134. FRET assays comprise the steps of exposing a sample portion comprising the donor located at a first position and the acceptor located at the second position to light at a first wavelength capable of inducing a first electronic transition in the donor, wherein the donor comprises a complex of lanthanide chelate and a lanthanide capable of binding the chelate and wherein the spectral overlap of the donor emission and acceptor absorption is sufficient to enable energy transfer from the donor to the acceptor as measured by a detectable increase in acceptor luminescence. Various coactivators for use in FRET assays have been described. Examples include, but are not limited to, Steroid Receptor Complex (SRC1), CREB binding protein (CBP), and Retinoid Interacting Protein (RIP 140). When a ligand binds to the ligand pocket of the receptor, the coactivator forms a receptor-coactivator complex. The current model on coactivators is that a ligand binds to the ligand binding domain (LBD) causing the activation function 2 (AF-2) to fold into place and trapping the ligand in the pocket. A novel interface (LXXLL motif) is formed by entrapment of the ligand, allowing the coactivator to interact with the AF-2. Thus, AF-2 is important in ligand dependent transactivation. When an inducer or agonist binds, it is transcriptionally active, while the binding of an inhibitor or antagonist interrupts the receptor cofactor interaction.

Cell free binding assays in which SHP, or the ligand binding domain of SHP (alone or present as a fusion protein), can also be performed. In these assays, the SHP or ligand binding domain of SHP is incubated with a test agent that, advantageously, bears a detectable label (e.g. a radioactive or fluorescent label). The SHP, or ligand binding domain thereof, free or bound to the test agent, is then separated from free test agent using any of variety of techniques (e.g., using gel filtration chromatography (for example, on Sephadex G50 spin columns) or through capture on a hydroxyapatite resin). The amount of test agent bound to the SHIP or ligand binding domain thereof, is then determined via detection of the label.

An alternative approach for detecting radiolabeled test agent bound to SHP, or ligand binding domain thereof, is a scintillation proximity assay (SPA). In this assay, a bead (or other particle) is impregnated with scintillant and coated with a molecule that can capture SHP, or the ligand binding domain thereof (e.g., streptavidin-coated beads can be used to capture biotinylated SHP ligand binding domain). Radioactive counts are detected only when the complex of radiolabeled test agent and the SHP, or ligand binding domain thereof, is captured on the surface of the SPA bead bringing the radioactive label into sufficient proximity to the scintillant to emit a signal. This approach has the advantage of not requiring the separation of free test agent from bound (Nichols et al, Anal. Biochem. 257:112-119 (1998)).

Assays to determine whether a test agent interacts with an SHP ligand binding domain can also be performed via a competition binding assay. In this assay, the SHP, or ligand binding domain thereof, is incubated with a compound known to interact with SHP, which compound, advantageously, bears a detectable label (e.g., a radioactive or fluorescent label). A test agent is added to the reaction and assayed for its ability to compete with the labeled compound for binding to SHP, or ligand binding domain thereof. A standard assay format employing a step to separate free known (labeled) compound from bound, or an SPA format, can be used to assess the ability of the test agent to compete.

To determine if a test agent activates SHP, thus inhibiting PEPCK, the ligand binding domain of SHP is prepared (e.g., expressed) as a fusion protein (e.g., with glutathione-S-transferase (GST), a histidine tag or a maltose binding protein). The fusion protein and coactivator (either or both advantageously labeled with a detectable label, e.g., a radiolabel or fluorescent tag) are incubated in the presence and absence of the test agent and the extent of binding of the coactivator to the fusion protein determined. The induction of interaction in the presence of the test agent is indicative of an activator of SHP.

SHP activation assays in accordance with the invention can be carried out using a full length SHP and a reporter system comprising one or more copies of the DNA binding site recognized by the SHP binding domain. More preferably, however, the activation assays are conducted using established chimeric receptor systems. For example, the ligand binding domain of SHP can be fused to the DNA binding domain of, for example, yeast transcription factor GAL4, or that of the estrogen or glucocorticoid receptor. An expression vector for the chimera (e.g., a GAL4-SHP chimera) can be transfected into host cells (e.g., CV-1, HuH7, HepG2 or Caco2 cells) together with a reported construct. The reporter construct can comprise one or more (e.g., 5) copies of the DNA binding site recognized by the binding domain present in the chimera (e.g., the GAL4 DNA binding site) driving expression of a reporter gene (e.g., CAT, SPAP or luciferase). Cells containing the constructs are then treated with either vehicle alone or vehicle containing test agent, and the level of expression of the reporter gene determined. In accordance with this assay, enhancement of expression of the reporter gene in the presence of the test agent indicates that the test agent activates SHP and thus can function as an inhibitor of glucose production.

Another format suitable for identifying ligands of SHP is the yeast two-hybrid assay. This is an established approach to detect protein-protein interactions that is performed in yeast. Protein # 1, representing the bait, is expressed in yeast as a chimera with a DNA binding domain (e.g., GAL4). Protein #2, representing the predator, is expressed in the same yeast cell as a chimera with a strong transcriptional activation domain. The interaction of bait and predator results in the activation of a reporter gene (e.g., luciferase or beta-galactosidase) or the regulation of a selectable marker (e.g., LEU2 gene). This approach can be used as a screen to detect, for example, ligand-dependent interactions between SHP and other proteins such as coactivator proteins (e.g., SRCI, TIFI, TIF2, ACTR) or fragments thereof (Fields et al., Nature 340:245-2.46 (1989)).

Suitable test agents that can be tested in the above assays include combinatorial libraries, defined chemical entities and compounds, peptide and peptide mimetics, oligonucleotides and natural product libraries, such as display (e.g. phage display libraries) and antibody products.

Typically, organic molecules are screened, preferably small organic molecules that have a molecular weight of from 50 to 2500 daltons. Candidate products are biomolecules including, saccharides, fatty acids, steroids, purines, pyrimidines, derivatives, structural analogs or combinations thereof. Such agents are obtained from a wide variety of sources including libraries of synthetic and natural compounds. Further, known pharmacological agents can be subjected to directed or random chemical modifications, such as acylation, alkylation, esterification, amidification, etc. to produce structural analogs.

Test agents can be used in an initial screen of, for example, 10 agents per reaction, and the agents of these batches that show inhibition or activation tested individually. Test agents may be used at a concentration of from 1 nM to 1000 μM, preferably from 1 μM to 100 μM, more preferably from 1 μM to 10 μM. Preferably, the activity of a test agent is compared to the activity shown by a known activator or inhibitor. A test agent that acts as an inhibitor preferably produces a 50% inhibition of activity of the receptor. Alternatively a test substance that acts as an activator preferably produces 50% of the maximal activity produced using a known activator.

Agents identified as modulators of SHP expression and/or activity can be administered to a subject to alter glucose production. Agents that induce or increase expression and/or activity of SHP decrease expression levels of PEPCK, thereby decreasing glucose production. Accordingly, compositions of the present invention comprising an agent that induces or increases SHP expression and/or activity are useful in decreasing glucose production in a subject and in the treatment of hyperglycemia. Such agents are useful in treating hyperglycemia resulting from, for example, obesity, impaired glucose tolerance, insulin resistance, metabolic syndrome X, Type 2 diabetes, Type 1 diabetes, and cardiovascular disease. Agents that inhibit or decrease expression and/or activity of SHP increase expression levels of PEPCK, thereby increasing glucose production. Compositions of the present invention comprising an agent that inhibits or decreases expression and/or activity of SHP are useful in increasing glucose in s a subject and in the treatment of hypoglycemia.

Dosing regimes, as well as selection of appropriate routes of administration for compositions comprising an agent of the present invention can be determined routinely by one of skill in the art based upon pharmacological activities of the agent in in vitro and in vivo assays such as described herein. It is preferred that compositions of the present invention comprise an amount of agent which is effective at modulating PEPCK expression levels so that production of glucose is altered. This effective amount can be determined routinely for each identified agent based upon its activity determined in vitro in screening assays such as described herein and in vivo in animal models. Effective amounts can be confirmed in subjects in need thereof by monitoring the effects of the agent on glucose levels in the subject. Methods for monitoring glucose levels in a subject are well known and performed routinely by those skilled in the art.

Agents of the present invention identified as modulators of SHP may be formulated into pharmaceutically acceptable compositions for administration to a subject by any route appropriate for modulation of glucose production. Suitable pharmaceutical formulations include, but are not limited to, those for oral, rectal, nasal, topical (including buccal and sublingual), vaginal or parenteral (including intramuscular, subcutaneous, intravenous, and directly into the affected tissue) administration or in a form suitable for administration by inhalation or insufflation. The formulation may, where appropriate be presented in convenient, discrete dosage units and may be prepared by any method well known in the art of pharmacy. All methods include the step of bringing into association the active agent with a liquid or finely divided solid carrier or both and then, if needed, shaping of the product into the desired formulation.

Pharmaceutical formulations suitable for oral administration may be presented in convenient discrete units including, but not limited to, capsules, cachets, or tablets, each containing a predetermined amount of the active agent; as a powder or granules; as a solution, a suspension or as an emulsion. The active agent can also be presented as a bolus, electuary, or paste. Tablets and capsules for oral administration may contain conventional excipients such as binding agents, fillers, lubricants, disintegrants, or wetting agents. The tablets may be coated according to methods well known in the art. Timed release formulations, which are known in the art, may also be suitable. Oral liquid preparations may be in the form of, for example, aqueous or oily suspensions, solutions, emulsions, syrups or elixirs, or may be presented as a dry product for constitution with water or other suitable vehicles before use. Such liquid preparations may contain conventional additives such as suspending agents, non-aqueous vehicles, including edible oils, or preservatives.

Agents of the present invention identified as modulators of SHP may also be formulated for parenteral administration, such as by injection, for example bolus injection or continuous infusion, and may be presented in unit dose form in ampules, pre-filled syringes, small volume infusion or in multi-dose containers with an added preservative. Pharmaceutically acceptable compositions comprising an active agent for parenteral administration may take the form of suspension, solution or emulsion in oily or aqueous vehicles, and may contain formulatory agents such as suspending, stabilizing, and/or dispersing agents. Alternatively, the active ingredient may be in powder form, obtained by asceptic isolation of sterile solid or by lyophilization from solution, for constitution with a suitable vehicle such as sterile, pyrogen free water, before use.

For topical administration to the epidermis, agents of the present invention identified as modulators of SHP may be formulated as ointments, creams, or lotions, or as a transdermal patch. Ointments and creams, may, for example, be formulated with an aqueous or oily base with the addition of suitable thickening and/or gelling agents. Lotions may be formulated with an aqueous or oily base and will in general also contain one or more emulsifying agents, stabilizing agents, suspending agents, thickening agents, or coloring agents. Formulations suitable for topical administration in the mouth include lozenges comprising an active agent in a flavored base, usually sucrose and acacia or tragacanth; pastilles comprising the active ingredient in an inert base such as gelatin and glycerin or sucrose and acacia; and mouth washes comprising the active ingredient in a suitable liquid carrier. For topical administration to the eye, the active agent can be made up in solution or suspension in a suitable sterile aqueous or non-aqueous vehicle. Additives such as buffers (e.g. sodium metabisulphite or disodium edeate) and thickening agents such as hypromellose can also be included.

Pharmaceutical formulations suitable for rectal administration wherein the carrier is a solid are preferably presented as unit dose suppositories. Suitable carriers include cocoa butter and other materials commonly used in the art, and the suppositories may be conveniently formed by admixture of the active agent with the softened or melted carrier or carriers followed by chilling and shaping in molds.

Formulations suitable for vaginal administration may be presented as pessaries, tampons, creams, gels, pastes, foams, or sprays containing in addition to the active agent such carriers as are known in the art to be appropriate.

For intra-nasal administration, agents of the present invention identified as modulators of SHP can be used as a liquid spray or dispersible powder or in the form of drops. Drops may be formulated with an aqueous or non-aqueous base also comprising one or more dispersing agents, solubilizing agents, or suspending agents. Liquid sprays are conveniently delivered from pressurized packs.

For administration by inhalation, agents of the present invention identified as modulators of SHP can be delivered by insufflator, nebulizer or a pressurized pack or other convenient means of delivering the aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the active agents of the present invention can take the form of a dry powder composition, for example a powder mix of an agent which modulates SHP and a suitable powder base such as lactose or starch. The powder composition may be presented in unit dosage form in, for example, capsules, cartridges or blister packs of gelatins, from which the powder can be administered with the aid of an inhalator or insufflator.

When desired, any of the above-described formulations may be adapted to provide sustained release of the agents of the present invention identified as modulators of SHP.

The pharmaceutical compositions of the present invention comprising an agent that modulates SHP expression and/or activity can also used in combination with other therapeutic agents.

The amount of an agent of the present invention required for use in treatment will of course vary with the route of administration, the nature of the condition being treated, and the age and condition of the subject being treated. Selection of such an amount, referred to herein as the “therapeutically effective amount or concentration” is ultimately at the discretion of the attending physician. In general, however, suitable doses of pharmaceutical compositions of the present invention providing a therapeutically effective amount of an agent which modulates SHP expression and/or activity will be in the range of from about 0.1 to 300 mg/kg of bodyweight per day, particularly from about 1 to 100 mg/kg of bodyweight per day. An appropriate dosage unit for oral administration generally contains from about 1 to about 250 mg, more preferably 25 to 250 mg of an active agent.

When used in the treatment of hyperglycemia, pharmaceutical compositions comprising an inducer of SHP expression and/or activity can be administered by any of the aforementioned routes, preferably by the oral route or by injection. The daily dosage for a 70 kg mammal will typically be in the range of about 5 mg to 5 grams of active agent of the present invention. Similar daily dosages of an inhibitor of SHP expression and/or activity can be administered for treatment of hypoglycemia.

The following nonlimiting examples are provided to further illustrate the present invention.

### EXAMPLES

### Example 1

### Insulin Treatment of ZDF Rats

All procedures performed were in compliance with the Animal Welfare Act, USDA regulations and approved by the GlaxoSmithKline Institutional Animal Care and Use Committee. Animals were housed at 72 F and 50% relative humidity with a 12 hour light and dark cycle, and fed chow diet (Formulab Diet 5008, PMI Feeds Inc., Richmond, Ind.). Insulin-treated animals received a mixture of HUMULIN®N and HUMULIN®R (Lilly, Indianapolis, Ind.) by subcutaneous injection and were sacrificed six hours later. RTQ-PCR was performed using an ABI PRISM 7700 Sequence Detection System instrument and software (PE Applied Biosystems, Inc., Foster City, Calif.) in accordance with procedures described by Way et al. (Endocrinology 2001 142: 1269-77). Primers used include:Rat PEPCK forward: TGAGGAAGTTTGTGGAAG (SEQ ID NO:1)
Rat PEPCK reverse: GCCGTCGCAGATGTGAAT (SEQ ID NO:2)
Rat PEPCK probe: TGCCCAGCTGTGCCAGCC (SEQ ID NO:3)
Rat SHP forward: TGGTACCCAGCTAGCCAAG (SEQ ID NO:4)
Rat SHP reverse: TGTTCTTGAGGGTGGAAGC (SEQ ID NO: 5)
Rat SHP probe: CGCCTGGCCCGAATCCTCC (SEQ ID NO:6)

[image]

### Example 2

### SHP Suppresses the Activity of the Glucocorticoid Receptor (GR)—a Known Positive Regulator of PEPCK and Gluconeogenesis, thereby Suppressing Glucose Biogenesis

A commercially available simian kidney-derived cell line (CV-1) was transfected with a GR-responsive luciferase reporter gene construct generated from the mouse mammary tumor virus (MMTV), a human GR expression vector, and, where indicated, a human SHP expression vector. To control for transfection efficiency, cells were co-transfected with pβ-actin-SPAP which contains the human secreted placental alkaline phosphatase cDNA linked to the β-actin promoter (Goodwin et al. Mol. Cell. 2000 6:517-26). Cells were cultured for 24 hours in the presence of a high-affinity GR ligand (dexamethasone at 1 μM) and harvested prior to the determination of luciferase reporter gene activity. Reporter gene activity was normalized to SPAP activity. Figure: Glucocorticoid receptor activity is suppressed by SHP.

[image]

1. A method for identifying test agents that alter gluconeogenesis or the production of glucose comprising determining a test agent's ability to modulate expression or activity of short heterodimer partner.

2. A composition for alteration of gluconeogenesis or glucose production, said composition comprising an agent which modulates short heterodimer partner expression or activity in a pharmaceutically acceptable formulation.

3. The composition of claim 2 wherein the agent induces or increases short heterodimer partner expression or activity.

4. The composition of claim 2 wherein the agent inhibits or decreases short heterodimer partner expression or activity.

5. A method for altering glucose production in a subject comprising administering to a subject in need thereof the composition of claim 2.

6. A method for decreasing glucose production in a subject comprising administering to a subject in need thereof the composition of claim 3.

7. The method of claim 6 wherein the subject is suffering from obesity, impaired glucose tolerance, insulin resistance, metabolic syndrome X, Type 2 diabetes, Type 1 diabetes, or cardiovascular disease.

8. A method for treating hyperglycemia in a subject comprising administering to a subject suffering from hyperglycemia the composition of claim 3.

9. A method for treating hypoglycemia in a subject comprising administering to a subject suffering from hypoglycemia the composition of claim 4.


SEQUENCE LISTING (6 sequences)

SEQ ID NO: 1 - DNA, 18 bp, rattus
tgaggaagtttgtggaag

SEQ ID NO: 2 - DNA, 18 bp, rattus
gccgtcgcagatgtgaat

SEQ ID NO: 3 - DNA, 18 bp, rattus
tgcccagctgtgccagcc

SEQ ID NO: 4 - DNA, 19 bp, rattus
tggtacccagctagccaag

SEQ ID NO: 5 - DNA, 19 bp, rattus
tgttcttgagggtggaagc

SEQ ID NO: 6 - DNA, 19 bp, rattus
cgcctggcccgaatcctcc